(19) United States
(12) Patent Application Publication
(10) Pub. No.: US 2003/0125647 A1
(43) Pub. Date: Jul. 3, 2003

(54) Electric massaging device

(76) Inventor: Chia-Lu Lin, Taichung (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/029,820

(22) Filed: Dec. 31, 2001

(51) Int. Cl. A61N 1/18

(52) U.S. Cl. 601/15

(57) ABSTRACT

An electric massaging device including a housing having an open side and an interior receiving space. A circuit board and a set of cells are disposed in the receiving space. The circuit board is powered by the cells to generate low-frequency current. The circuit board has a controlling section inlaid in the housing, whereby a user is able to control the circuit board on outer side of the housing. Two electrodes protrude from the circuit board to the open side of the housing. The open side of the housing is connected with a conductive massaging plate covering the receiving space. The two electrodes of the circuit board contact with the conductive massaging plate.

### BACKGROUND OF THE INVENTION

The present invention is related to an electric massaging device in which a conductive massaging plate is directly connected with a housing of the electric massaging device, enabling a user to conveniently use the electric massaging device.

FIG. 6 shows a conventional electric massaging device 7 which includes a conductive rubber 71 having a fixing seat 72. One terminal of a conductive wire 73 is connected with the fixing seat 72. The other terminal of the conductive wire 73 is provided with a plug 74 plugged in a controlling box 75. The conductive rubber 71 is attached to human body. The conductive wire 73 conducts the current generated by the controlling box 75 to the conductive rubber 71 for massaging human body.

The above electric massaging device 7 includes multiple components and the conductive rubber 71 must be connected with the controlling box 75 via the conductive wire 73. It is inconvenient for a user to use such electric massaging device.

Moreover, in both use and storage, the outward extending conductive wire 73 is very easy to tangle with other articles. Furthermore, the conductive wire 73 and the plug 74 tend to loosen from the aperture 721 of the fixing seat 72 and the socket after repeatedly inserted and extracted.

In the case that the conductive wire 73 and the plug 74 are omitted and the electrode fastener (not shown ) on the controlling box 75 is directly attached to human body, the current will directly flow from the controlling box 75 through the electrode fastener to human body. However, the electrode fastener has smaller area and the user's skin will feel stung and uncomfortable.

FIG. 7 shows another type of electric massaging device 8 including a fastening belt 81 on which multiple conductive massaging cells 82 are disposed. The massaging cells 82 are connected with each other by conductive wires (not shown ). Each massaging cell 82 is equipped with an electrode fastener, whereby the massaging cell 82 can be connected with a low-frequency or midfrequency wave generator to create massaging effect.

When only a small area of a user's body (such as wrist and shoulder ) needs to be massaged, the fastening belt 81 with a certain length will be a burden to the user.

### SUMMARY OF THE INVENTION

It is therefore a primary object of the present invention to provide an electric massaging device which can be conveniently applied to any part of a user's body to massage the part without limitation of the configuration of the part. A conductive massaging plate is directly mounted on an open side of the housing of the electric massaging device. A user can conveniently place the electric massaging device on the shoulder, wrist, leg, etc. of his/her body to provide massaging effect.

It is a further object of the present invention to provide the above electric massaging device in which a circuit board and a set of cells are disposed in the interior receiving space of the housing. The open side of the housing is connected with a conductive massaging plate covering the receiving space. The electrodes of the circuit board directly contact with the conductive massaging plate. Therefore, no conductive wire or socket outward extends from the housing and problem of tangling of the wire with other articles is avoided.

It is still a further object of the present invention to provide the above electric massaging device which itself includes a conductive massaging plate. The electric massaging device can further include a fastening belt formed with multiple fixing holes corresponding to the profile of the housing of the electric massaging device for disposing the electric massaging device therein. A user can bind the fastening belt on his/her body to use the electric massaging device. Alternatively, the user can solely use the electric massaging device. The user can mount the electric massaging devices in different fixing holes of the fastening belt as necessary and adjust the number of the electric massaging devices on the fastening belt. Accordingly, the application of the electric massaging device is more variable.

The present invention can be best understood through the following description and accompanying drawings wherein:

### BRIEF DESCRIPTION OF THE DRAWINGS

FIG. 1 is a perspective assembled view of the present invention;

FIG. 2 is a sectional view taken along line 2-2 of FIG. 1;

FIG. 3 is a perspective view of a second embodiment of the present invention;

FIG. 4 shows that the electric massaging device of the present invention is to be mounted on a fastening belt;

FIG. 5 shows that the electric massaging device of the present invention is mounted on a fastening belt;

FIG. 6 is a perspective view of a conventional electric massaging device; and

FIG. 7 is a perspective view of another type of conventional electric massaging device.

### DETAILED DESCRIPTION OF THE PREFERRED EMBODIMENTS

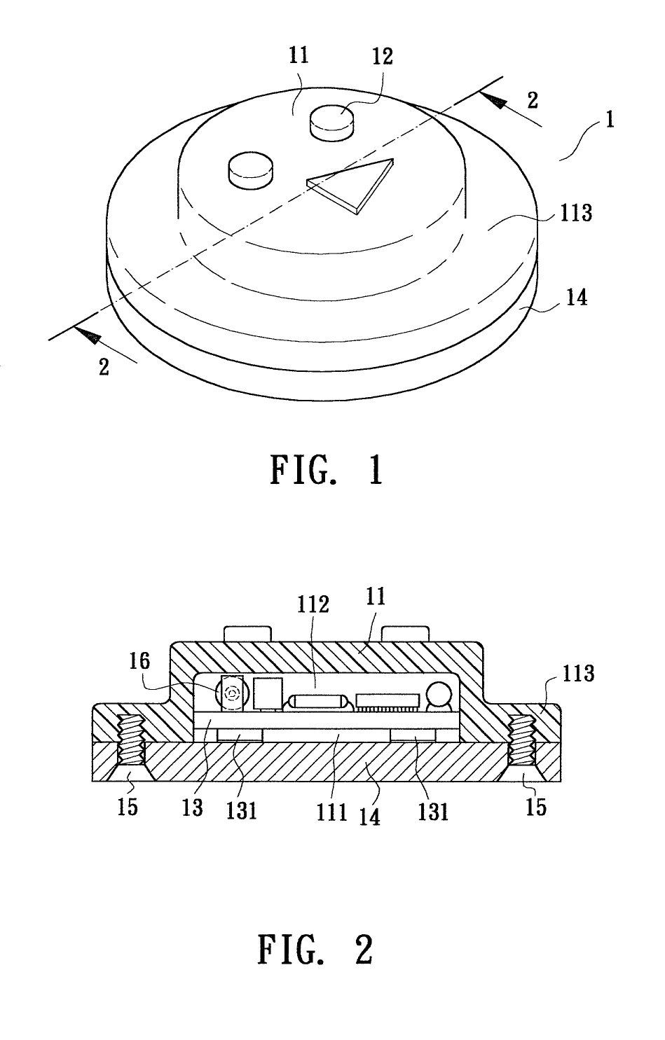

Please refer to FIGS. 1 and 2. The electric massaging device 1 of the present invention includes a housing 11 having an open side 111 and an interior receiving space 112. A circuit board 13 and a set of cells 16 are disposed in the receiving space 112. The circuit board 13 is powered by the cells 16 and has a controlling section 12 inlaid in the housing 11. A user can control the circuit board 13 on outer side of the housing 11. Two electrodes 131 protrude from the circuit board 13 to the open side 111 of the housing 11.

The bottom of the housing 11 is formed with an outward extending flange 113. The open side 111 of the housing 11 is connected with a conductive massaging plate covering the receiving space 112. In this embodiment, the conductive massaging plate is a conductive rubber 14 which also covers the flange 113. Two bolts 15 are passed through the conductive rubber 14 and screwed into the flange 113 to lock the conductive rubber 14 on the housing 11. The two electrodes 131 of the circuit board 13 contact with the conductive rubber 14.

In use, the conductive rubber 14 is attached to a part such as the leg, shoulder, etc. of a user's body to be massaged. The current generated by the circuit board 13 via the electrodes 131 is conducted to the conductive rubber 14 to provide a massaging effect for human body.

According to the above arrangement, the conductive rubber 14 directly mounted on the open side 111 of the housing 11 contacts with human body. The circuit board 13 is disposed in the receiving space 112 of the housing 11. A user can conveniently place the electric massaging device 1 on any part (such as shoulder, wrist, leg, etc. ) of his/her body to massage the part without limitation of the configuration of the part. The conductive rubber 14 contacts with the human body without stinging the skin.

In addition, the electrodes 131 of the circuit board 13 directly contact with the conductive rubber 14 and the controlling section 12 of the circuit board 13 is inlaid in the housing 11. Therefore, a user can control the circuit board 13 on outer side of the housing 11. Accordingly, it is no more necessary to arrange any conductive wire, plug or controlling box on outer side of the housing 11 and thus the problem of tangling of the conductive wire is avoided.

It should be noted that the conductive massaging plate can be made of conductive EVA or any other material used in this field.

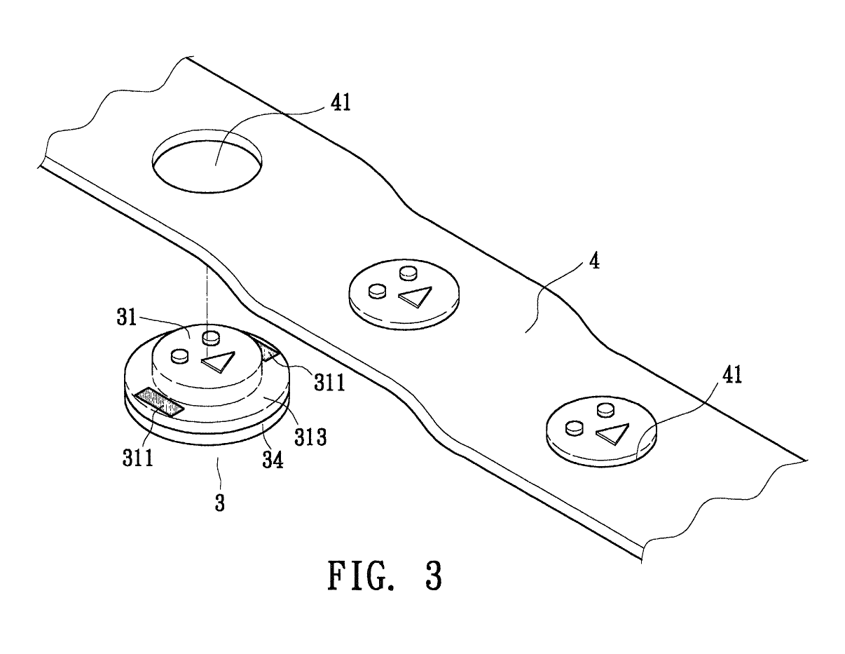

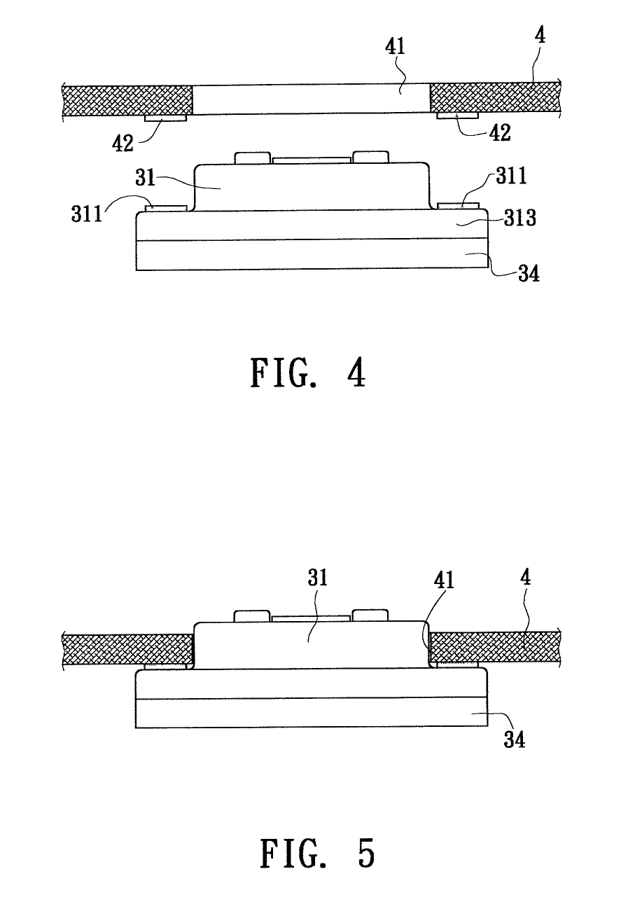

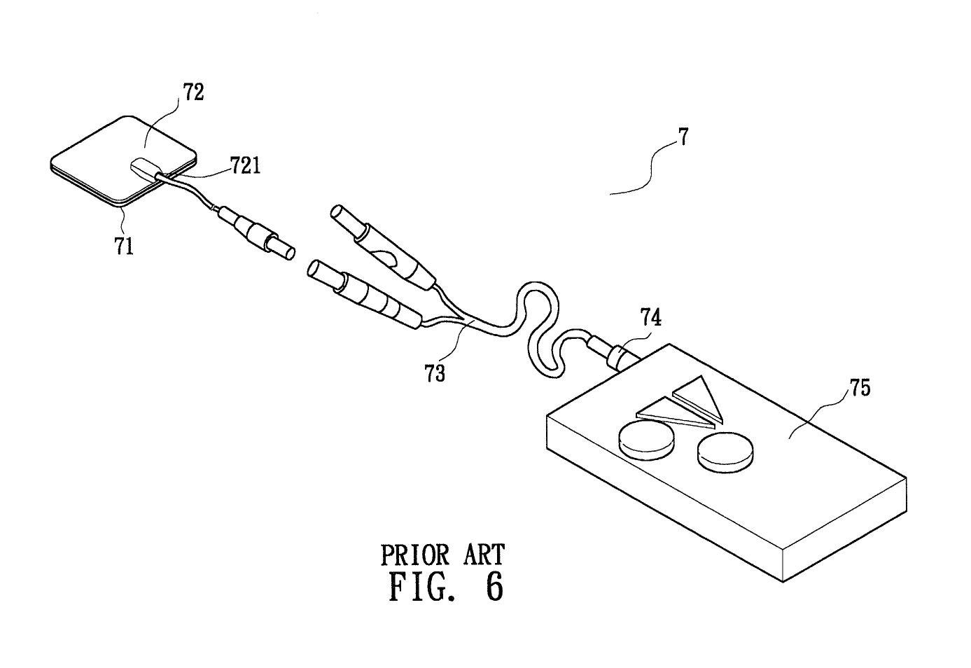

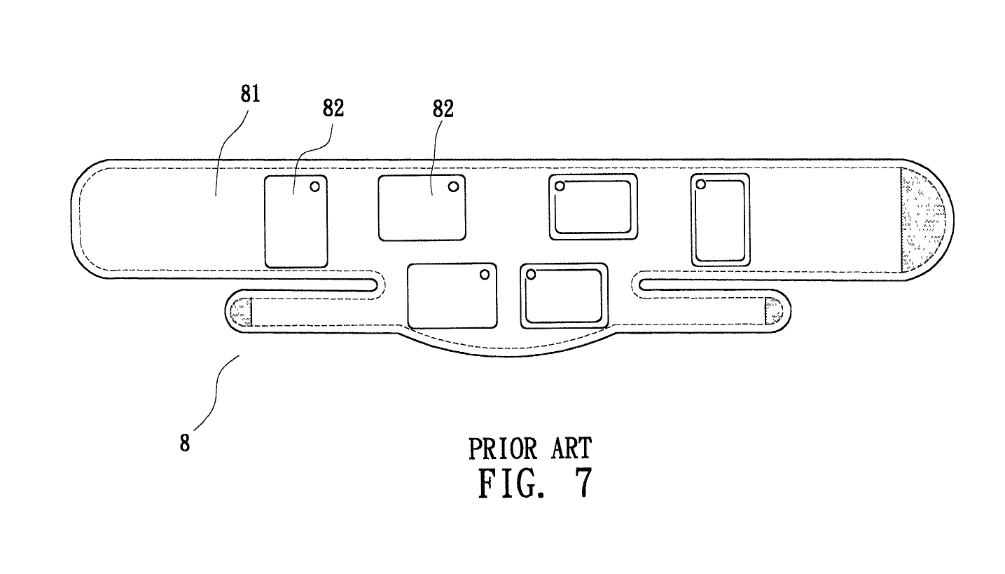

FIGS. 3 to 5 show a second embodiment of the present invention, which further includes a fastening belt 4 formed with multiple fixing holes 41 corresponding to the profile of the housing 31 of the electric massaging device for disposing the electric massaging device 3 therein. Two ends of the fastening belt 4 are provided with cooperative fasteners (not shown ) for fastening the fastening belt 4 on a user.

One face of the flange 313 of the housing 31 distal from the conductive massaging plate 34 is provided with a male magic tape 311. The fastening belt 4 is provided with at least one female magic tape 42 corresponding to the male magic tape 311. The male magic tape 311 is attached to the female magic tape 42 with the housing 31 fitted through the through hole of the fastening belt 4 to fix the electric massaging device 3 on the fastening belt 4.

In use, the fastening belt 4 is bound around a user's waist, thigh, etc. with the conductive massaging plate 34 attaching to the human body to provide massaging effect for the user. Certainly, the electric massaging device 3 can be also directly solely used without being mounted on the fastening belt 4.

Alternatively, a user can mount the electric massaging devices 3 in different fixing holes 41 of the fastening belt 4 as necessary or adjust the number of the electric massaging devices 3 on the fastening belt 4. Accordingly, the application of the electric massaging device 3 of the present invention is more variable.

The above embodiments are only used to illustrate the present invention, not intended to limit the scope thereof. Many modifications of the above embodiments can be made without departing from the spirit of the present invention.

What is claimed is:

1. An electric massaging device comprising a housing having an open side and an interior receiving space, a circuit board and a set of cells being disposed in the receiving space, the circuit board being powered by the cells to generate low-frequency current, the circuit board having a controlling section inlaid in the housing, whereby a user is able to control the circuit board on outer side of the housing, two electrodes protruding from the circuit board to the open side of the housing, the open side of the housing being connected with a conductive massaging plate covering the receiving space, the two electrodes of the circuit board contacting with the conductive massaging plate.

2. The electric massaging device as claimed in claim 1, wherein the conductive massaging plate is a conductive rubber.

3. The electric massaging device as claimed in claim 1, wherein the conductive massaging plate is a conductive EVA.

4. The electric massaging device as claimed in claim 1, further comprising a fastening belt formed with multiple fixing holes corresponding to the profile of the housing of the electric massaging device for disposing the electric massaging device therein, two ends of the fastening belt being provided with cooperative fasteners for fastening the fastening belt on a user.

5. The electric massaging device as claimed in claim 1, wherein the housing is provided with a male magic tape and the fastening belt is provided with at least one female magic tape corresponding to the male magic tape, whereby the male magic tape is attached to the female magic tape to fix the electric massaging device on the fastening belt.
